(19) United States
(12) Patent Application Publication
(10) Pub. No.: US 2005/0049649 A1
(43) Pub. Date: Mar. 3, 2005

(54) Electrical stimulation of the brain

(75) Inventors: Jurgen Luders, Cleveland Heights, OH (US); Imad Najm, Broadview Heights, OH (US); Hans O. Luders, Cleveland Heights, OH (US)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: The Cleveland Clinic Foundation

(21) Appl. No.: 10/689,455

(22) Filed: Oct. 20, 2003

Related U.S. Application Data
(60) Provisional application No. 60/420,079, filed on Oct. 21, 2002.

(51) Int. Cl. A61N 1/18

(52) U.S. Cl. 607/45

(57) ABSTRACT

The present invention relates electrical stimulation of structures having high fiber density, such as white matter tracts of the brain, to effect desired stimulation of associated brain structures. The stimulation of such white matter tracts or other structures can help reduce seizures or otherwise help control seizures by overdriving at least some electrical activity of the associated brain structures.

### RELATED APPLICATION

This application claims priority from U.S. provisional patent application Ser. No. 60/420,079, filed on Oct. 21, 2002, the subject matter of which is incorporated herein by reference.

### TECHNICAL FIELD

The present invention relates to treatment for the nervous system, and more particularly to systems and methods for electrically stimulating the brain.

### BACKGROUND OF INVENTION

Presently, various different approaches exist to electrically stimulate the brain to help alleviate degenerative diseases and nervous system disorders, such as Parkinson's disease and epilepsy. For example, electrical stimulation can provide an effective treatment for patients when surgical lesioning of brain tissue is not a suitable option as well as when patients are not sufficiently responsive to other treatment modalities, such as drug therapy.

Some different types of electrical stimulation treatments include vagal nerve stimulation, cerebellar stimulation, and deep brain stimulation. One major advantage of electrical stimulation over lesioning procedures (e.g., pallidotomy and thalamotomy) is that the electrical stimulation can be reversible and adjustable. For example, brain stimulation can be implemented with no destruction of brain tissue and the stimulator can be removed, if needed. Additionally, the stimulation can be adjusted (e.g., increased, minimized or turned off or otherwise modified) to achieve better clinical effects for each patient.

Vagal nerve stimulation is one accepted type of treatment for epilepsy and Parkinson's disease. Vagal nerve stimulation is typically performed via a stimulator device, which includes a generator that electrically stimulates the brain through the vagus nerve to prevent seizures. The generator is surgically implanted into the chest, such as under the collarbone, and can be activated automatically or manually, such as by passing a magnet over the device.

In general, deep brain nuclei stimulation involves the precise electrical stimulation of specific deep brain structures using implanted electrodes. Recently, there has been significant work in the area of electrical stimulation of the subthalamic nucleus (STN) in which miniature electrodes are placed into the STN on one or both sides of the brain. STN is a structure located deep within the brain that has been found to control many aspects of normal motor function. Electrical stimulation of the STN effectively jams or blocks the abnormal circuitry of the brain, such as in the case of Parkinson's disease or epilepsy.

### SUMMARY OF INVENTION

The following presents a simplified summary of the invention in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

The present invention relates to electrical stimulation of white matter tracts in the brain to mitigate or help control seizures. One or more stimulators or electrodes can be positioned to electrically stimulate a white matter tract to enable stimulation of an associated epileptogenic focus or zone. The particular white matter structure to which the stimulation is applied can vary based on the location of the epileptogenic zone.

According to one aspect of the present invention, electrical stimulation can be applied to the fornix, such as where the epileptic zone has been determined to include the hippocampus. According to another aspect of the present invention, electrical stimulation can be applied to the corpus callosum, such as where the epileptic zone has been determined to be cortical.

Various types of stimulators can be utilized to electrically stimulate desired white matter according to an aspect of the present invention. By way of example, the stimulator can include a generally annular or C-shaped body that can circumscribe at least a portion of the desired white matter, such as the body of the fornix, which is associated with the epileptogenic zone. The body portion includes one or more electrodes that can electrically stimulate the desired white matter when implanted. Alternatively, the stimulator can be implanted into the white matter itself. The stimulator is adapted to receive an electrical signal from a signal generator for stimulating the white matter in a desired matter. The signal generator can be programmable. To facilitate implantation of the stimulator, endoscopic means can be efficiently utilized to associate the stimulator with desired white matter in accordance with an aspect of the present invention.

### BRIEF DESCRIPTION OF THE DRAWINGS

The foregoing and other aspects of the present invention will become apparent to those skilled in the art to which the present invention relates upon reading the following description with reference to the accompanying drawings.

FIG. 1 is a block diagram illustrating a brain stimulation system in accordance with an aspect of the present invention.

FIG. 2 is an example of one type of brain stimulation system for stimulating the fornix in accordance with an aspect of the present invention.

FIG. 3 is another view of a brain stimulator located for stimulating the fornix associated with an epileptogenic structure in accordance with an aspect of the present invention.

FIG. 3A depicts some neurological pathways associated with the hippocampus and fornix that can be employed in stimulation in accordance with an aspect of the present invention.

FIG. 4 is an example of a stimulator device in accordance with an aspect of the present invention.

FIG. 5 is an example of another type of brain stimulation system for stimulating the fornix in accordance with an aspect of the present invention.

FIG. 6 is an example of one type of brain stimulation system for stimulating the corpus callosum in accordance with an aspect of the present invention.

FIG. 7 is a coronal section of the brain illustrating corpus callosum stimulation in accordance with an aspect of the present invention.

FIG. 8 is an example of another type of brain stimulation system for stimulating the corpus callosum in accordance with an aspect of the present invention.

FIG. 9 is a flow diagram illustrating a methodology for brain stimulation in accordance with an aspect of the present invention.

### DETAILED DESCRIPTION OF THE INVENTION

The present invention relates electrical stimulation of structures having high fiber density, such as white matter tracts, to effect desired stimulation of associated epileptogenic structures. The stimulation of such white matter tracts or other structures can help reduce seizures or otherwise help control seizures by electrically overdriving the associated epileptogenic structures.

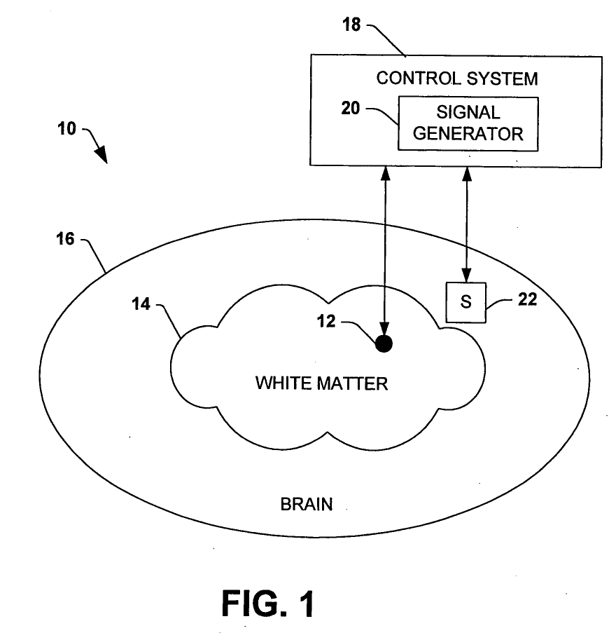

FIG. 1 depicts a schematic example of a brain stimulation system 10 in accordance with an aspect of the present invention. The system 10 includes a stimulator 12 electrically associated with a white matter tract 14 of a patient's brain 16. White matter is generally formed of nerve fibers, called axons, which are insulated by a fatty substance known as myelin. White matter carries information between nerve cells of associated non-white matter brain structures by conducting electrical impulses through the nerve fibers.

Various configurations of stimulators can be utilized for white matter stimulation in accordance with an aspect of the present invention. For example, the stimulator 12 can be configured as an elongated rod, a depth electrode, a ring, a clamp, or other devices capable of providing desired electrical stimulation to the white matter 14. The stimulator 12 can be self-contained and include a signal generator or, alternatively, it may receive electrical signals from a control system 18. According to one particular aspect, the stimulator 12 can be collapsible or otherwise deformable to facilitate endoscopic implantation of the stimulator.

A control system 18 is operative to control operation of the stimulator 12, such as by providing a signal to the stimulator for electrically stimulating the white matter tract 14 based on the signal. For example, the control system 18 can be coupled to the stimulator 12 through an electrically conductive element, which provides an electrical signal having desired electrical characteristics. Alternatively or additionally, the control system 18 can be configured to activate the stimulator via wireless means, such as electromagnetic fields (e.g., radio frequency (RF)), magnetic fields and the like to provide desired stimulation. That is, a direct connection between the control system 18 and the stimulator 12 is not required.

The control system 18 can include a signal generator 20 programmed and/or configured to activate the stimulator for white matter stimulation at a desired intensity (e.g., amperage) and frequency over a predetermined time period. For example, the signal generator can provide electrical pulses at a frequency ranging from about 0.1 Hz to about 5000 Hz. It has been determined some patient's may respond better to low frequency stimulation, such as at a frequency less than about 10 Hz (e.g., in a range from about 0.5 Hz to about 4 Hz). The duty cycle (or pulse width) of such pulses also can be programmable. The amplitude of electrical current also may vary based at least in part on the patient's condition and the white matter 14 structure to which the stimulator is positioned. For example, the signal generator 20 can be configured to provide electrical current having an amplitude in a range from 0 to about 5 mA, which can be a monophase or polyphase signal.

The stimulator 12 is positioned (e.g., by stereotaxis or endoscopy) relative to the white matter tract 14 to enable desired electrical stimulation of a corresponding epileptogenic focus in response to activation of the stimulator. The white matter 14 is fibrous connection that provides an electrical pathway between the stimulator 12 and the corresponding epileptogenic focus. The stimulator 12 can be positioned adjacent to, in contact with or within a selected white matter structure 12. Where more than one epileptogenic focus exists, multiple stimulators can be utilized to stimulate white matter structures associated with each respective focus in accordance with an aspect of the present invention. For example, stimulators can be used unilaterally, such as where a focus exists only in a single hemisphere of the brain 16, or bilaterally, such as where foci exist in both hemispheres.

Various diagnostic techniques can be utilized, individually or in combination, to determine the location of one or more epileptogenic foci (or zones) for a patient. Some examples include electroencephalography (EEG), magnetic resonance imaging (MRI), positron emission tomography (PET), single photon emission computed tomography (SPECT), magnetoencephalography (MEG), magnetic resonance spectroscopy (MRS), depth electrode and subdural grid implantation, video monitoring, neuropsychological testing and so forth. Those skilled in the art will understand and appreciate other types of diagnostic techniques that can be utilized to help ascertain epileptogenic zones in a patient.

By way of example, some epileptogenic structures include the hippocampus and neocortical structures. According to an aspect of the present invention, the stimulator 12 can be positioned to stimulate these and other epileptogenic structures by direct electrical stimulation of corresponding white matter tracts, at least a portion of which are connected to such epileptogenic structures. For example, fornix stimulation can be utilized in the case of epileptoid convulsions originating in the hippocampus. The fornix is the white matter tract that is a major output pathway of each hippocampal formation, connecting it to the frontal lobe, and parts of thalamus and hypothalamus. Stimulation of the corpus callosum, for example, can be employed for stimulating neocortical areas. The corpus callosum comprises the white matter bundles which collectively serve to interconnect cortical areas in the two cerebral hemispheres of the brain 16.

Those skilled in the art will understand and appreciate that other white matter tracts (e.g., the temporal stem) can be utilized to overdrive electrical activity in other epileptic zones (e.g., lateral or temporal lobes). As mentioned above, one or more stimulators can be used to stimulate appropriate white matter, such as unilaterally or bilaterally, depending on the epileptogenic zone or zones.

By way of further example, the brain stimulation system 10 can be implemented as a closed loop system in which the control system 18 is operative to activate the stimulator 12 in response a sensed characteristic of the brain 16. For example, one or more sensors 22 can be used to sense electrical activity associated with the onset of a seizure or other neurological condition. The sensors 22 can be subdural or external probes located at or near the determined epileptogenic zones. Alternatively or additionally, the stimulator 12 itself can be configured to operate as a sensor and provide signals to the control system 18 indicative of seizure onset. The control system 18 thus can control the signal generator 20 to operate the stimulator 12 to provide stimulation as a function of sensed electrical (or chemical) activity of the brain 16. The stimulation can include electrical current having an amplitude, frequency and pulse width, some or all of which can vary based on the sensed characteristic(s). Those skilled in the art will understand and appreciate various types of sensors and detection software that can be utilized to detect seizure onset, all of which can be employed to control stimulation in accordance with an aspect of the present invention.

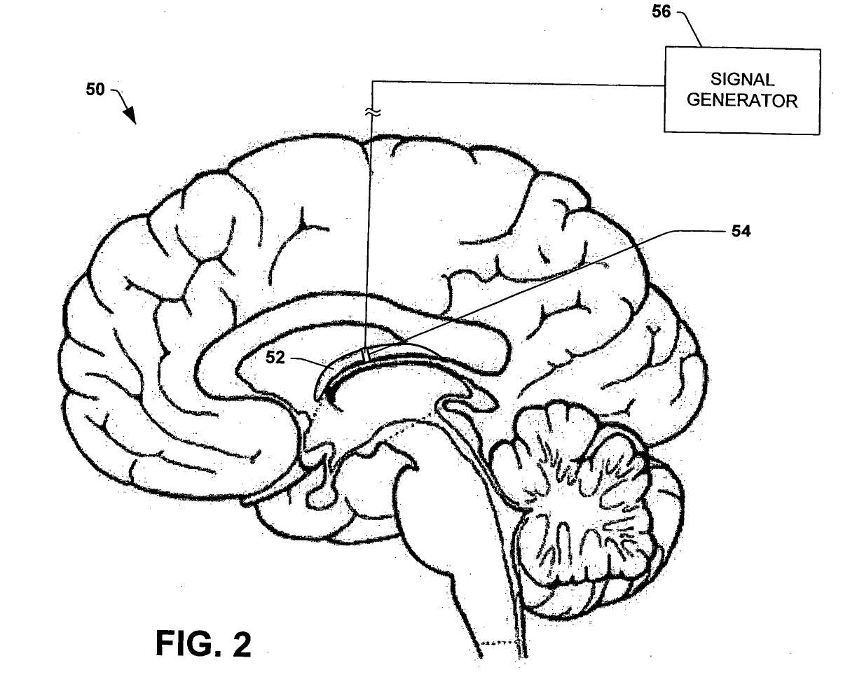

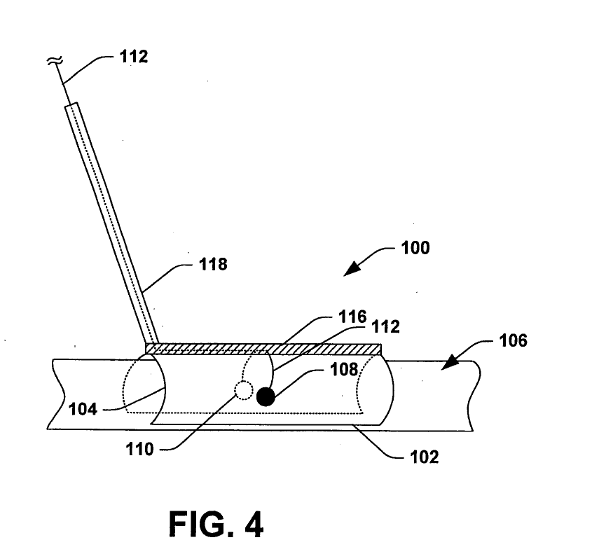

FIG. 2 is an example of an electrical stimulation system 50 implemented to stimulate the fornix 52 in accordance with an aspect of the present invention. In this example, the system 50 includes a stimulator 54 that is positioned at or around at least part of the body of the fornix 52, such as for corresponding hippocampal stimulation. For example, the stimulator 54 can include one or more electrodes that contact the fornix 52 for providing electrical stimulation according to electrical signals from an associated electrical signal generator 56. Such a stimulator 54 electrode can be implemented as a depth electrode or it can be attached to a support, which can be a clamp, pronged adaptor, or frame configured for relatively secure attachment to the fornix (See, e.g., FIG. 4).

Those skilled in the art will understand and appreciate that the stimulator position for the fornix 52 may vary from patient to patient as well as based on the determined location of the epileptogenic focus. The stimulator 54 can be positioned stereotactically or endoscopically. Endoscopy is particularly useful for positioning the stimulator at the fornix, as the fornix is accessible through corresponding lateral ventricles. Endoscopy thus facilitates implantation of the stimulator 54 aided by its visual component.

The signal generator 56 can be programmable to control operation of the stimulator 54 in a desired manner. For example, the signal generator 56 can be programmed to activate the stimulator 54 intermittently or periodically to provide electrical current to the fornix 52 at a desired intensity (e.g., amperage), frequency, and duty cycle over a predetermined time period. By way of further example, the signal generator 56 can form part of a closed loop system that includes a controller operative to activate the stimulator 54 in response to sensing onset of a seizure or other neurological disorders. Such a system can employ one or more other sensors (e.g., subdural probes) located at or near the determined epileptogenic zones. Alternatively or additionally, the stimulator 54 itself can be configured to operate as a sensor and provide signals to the control system indicative of seizure onset. The control system thus can control the stimulator to provide stimulation with electrical current having electrical characteristics (e.g., amplitude, frequency, ON/OFF times, and duty cycle) that can vary based on the sensed onset, such as described herein.

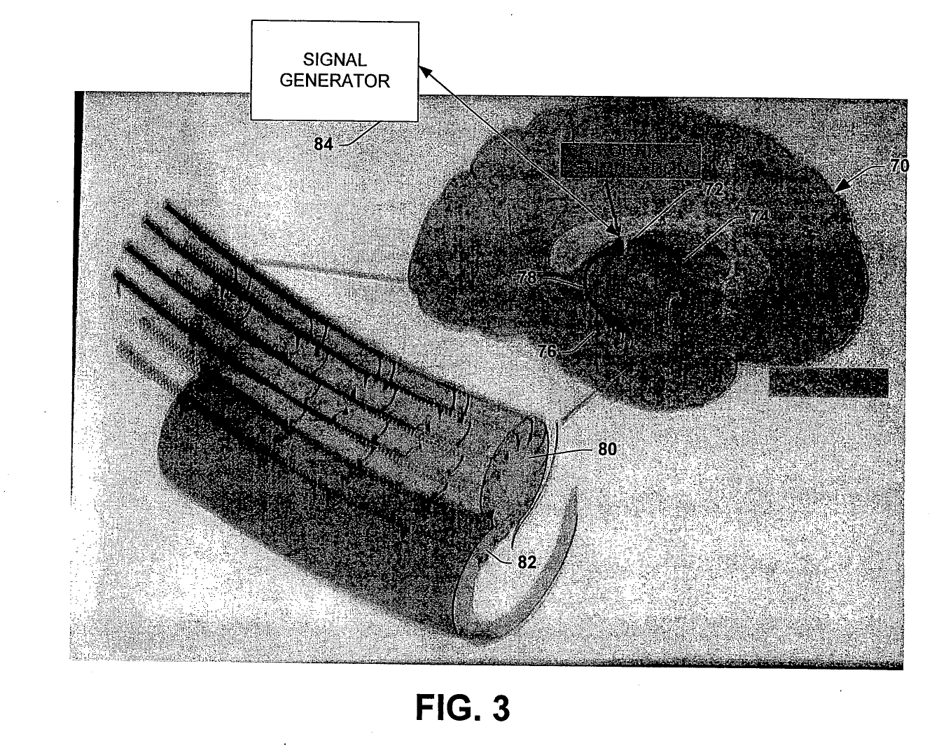

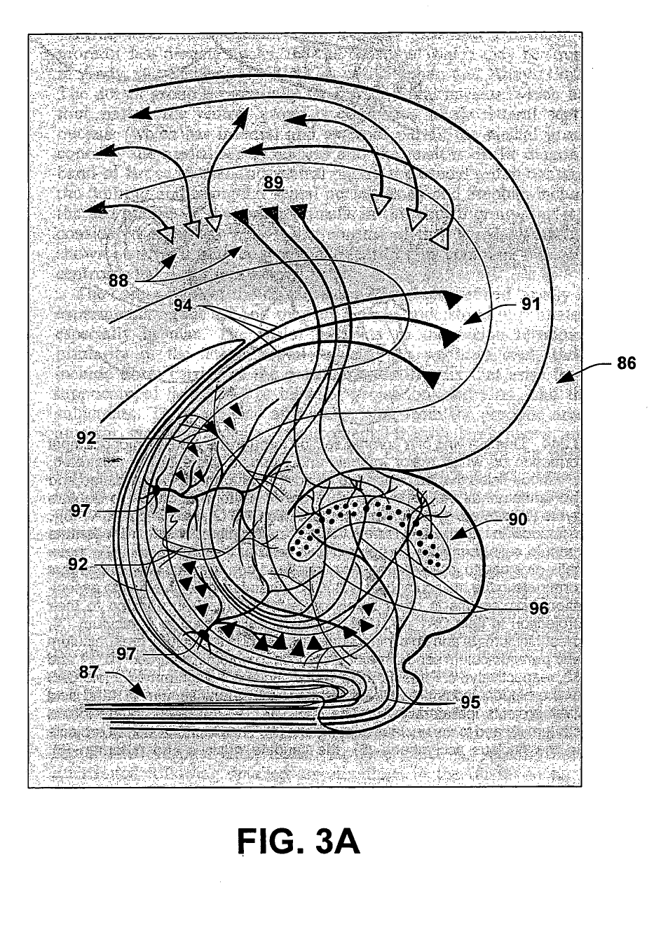

FIG. 3 depicts another example of fornix stimulation in accordance with an aspect of the present invention. In this example, an annular stimulator 72 has been implanted around part of the fornix 74, such as at the fornix body spaced apart from the hippocampus 76. The fornix 74 includes numerous neuron fibers, schematically represented at 78. Approximately 50% of the fornix fibers 78, which includes fibers for both orthodromic and antidromic impulses, connect the hippocampus 76 with the hyphothalamus (not shown). Such fibers also form part of the circuit of Papez. Because the fornix fibers 78 connect to the hippocampus 76, such fibers provide an efficient electrical pathway for transferring electrical stimulation from the fornix 74 to the hippocampus 76. The enlarged part of FIG. 3 further diagrammatically represents the transfer of electrical signals at the juncture between the crura of fornix 80 to the fimbria of hippocampus 82, which hippocampal stimulation varies based on the fornix stimulation.

Those skilled in the art will understand and appreciate that such fornix stimulation via one or more stimulators 72 can provide effective seizure control at focal areas (e.g., the hippocampus 76) directly connected with the associated fibers 78. The operation of the stimulator 72 can be controlled by electrical signals provided by an associated signal generator 84, which can be located intra-cranially (e.g., subdurally) or at least a portion of the generator can be exteriorized from the patient.

FIG. 3A is schematic representation of the major information pathways in the hippocampal region 86 and their relationship with the fornix 87. From this figure, those skilled in the art will appreciate the types of electrical pathways that can be utilized to enable overdriving epileptogenic zones in the hippocampal region by fornix stimulation in accordance with an aspect of the present invention.

As represented in FIG. 3A, the perforant pathway 88 carries output from the superficial entorhinal cortex 89, which forms the input to the hippocampus and is responsible for the pre-processing of input signals. The perforant pathways 88 carry signals from the entorhinal cortex 89 to the dentate gyrus 90, and information travels thence to fields CA1-CA4, the subiculum 91, and back to the deep layers of the entorhinal cortex, which, in turn, sends output back to the sensory association areas. Also depicted are efferents 92 from pyramidal cells 93 in hippocampal fields CA1-CA4. Efferents 94 from the subiculum 91 are also associated with the fornix 87. Afferents 95 from the fornix 87 also are shown as terminating in the mossy fibers 96 of the dentate gyrus 90. The mossy fibers 96 branch profusely in white matter structures, each branch having multiple swellings that contain round vesicles and synaptic thickenings. Basket cells 97 further are illustrated, which inhibit the piriform neurons, which, in turn, neurons inhibit the deep nuclei and the vestibular nuclei on which their axons synapse.

FIG. 4 illustrates an example of a stimulator device 100 in accordance with an aspect of the present invention. The device 100 includes a generally elongated body portion 102 dimensioned and configured for attaching to desired white matter structure. In the example of FIG. 4, the body portion 102 is depicted as a generally cylindrical portion having an inner sidewall portion 104 configured for attachment to white matter, such as the fornix 106. The body portion 102 also can be deformable to a reduced cross-sectional dimension, such as to facilitate its implantation and positioning around the fornix 106.

By way of example, the body portion 102 can be formed of a substantially resilient flexible material that can be urged into a tubular structure, such as for implantation via endoscopy. In this way, the stimulator device 100 can be inserted into an endoscope (not shown) for implantation through a small cranial burr-hole. A distal end of the endoscope can be guided through a ventricle (e.g. right or left lateral ventricles) so as to facilitate positioning the device 100 around the corresponding fornix 106. For example, within the endoscope, the device has a reduced cross-sectional dimension to facilitate its implantation. Once the distal end of the endoscope is sufficiently near the fornix 106, the device 100 can be expanded to its expanded dimension (e.g., spring activated, urged open by balloon catheterization) for attachment to the fornix 106, such as depicted in FIG. 4.

One or more electrodes 108 and 110 are disposed along the body portion 102 for providing electrical current to the fornix 106. While two electrodes are shown in FIG. 4, those skilled in the art will appreciate that any number of one or more electrodes having any desired configuration (e.g., circular, rectangular) can be utilized in accordance with an aspect of the present invention. The electrodes 108 and 110 can be mounted to the interior sidewall 104 or be otherwise attached thereto.

The electrodes 108 and 110 are electrically coupled to receive corresponding electrical signals from one or more sources of electrical energy, such as a signal generator (not shown). For example, electrical conductors 112 can extend from the electrodes 108, 110 to within a base plate 116 and through an insulating structure 118, such as a tube formed of an insulating material. The plate 116 can be attached to the body portion 102 or it can be formed integrally with the body portion. The plate 116 and tube 116 can be formed of the same or different material. Additionally or alternatively, the body portion 102 can be formed of the same or a different material from the base plate 116. Those skilled in the art will understand and appreciate various types of materials that can be used to form the various parts of the device 100 based on the above description, all of which are contemplated as falling within the spirit and scope of the present invention.

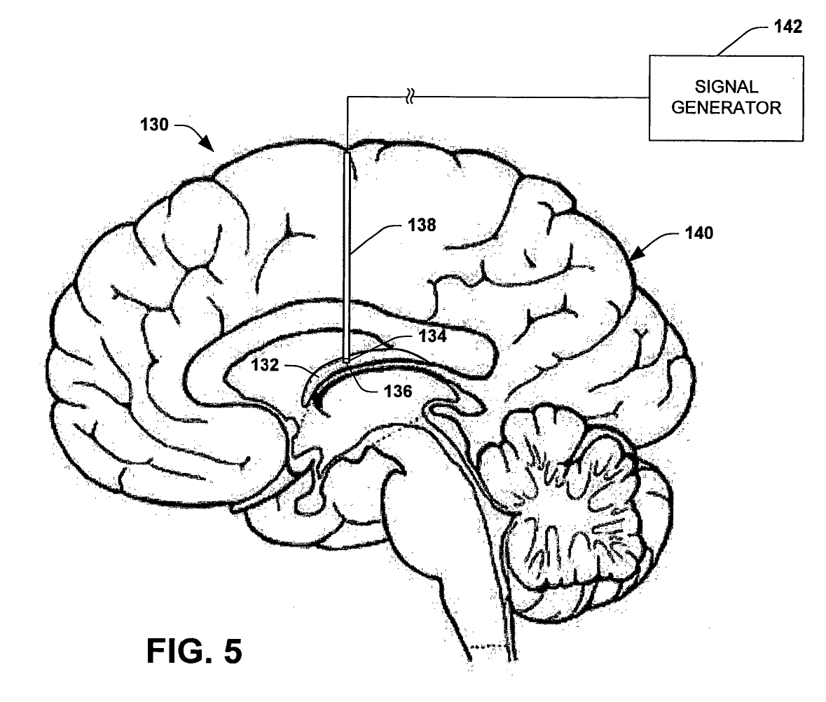

FIG. 5 is an example of another electrical stimulation system 130 that can be employed for fornix 132 stimulation in accordance with an aspect of the present invention. In this example, the system 130 includes a stimulator 134 that is located at or near an end 136 of an elongated rod 138. The rod 138 is inserted into the brain 140 in a minimally invasive manner to position the stimulator 134 in contact with, inside or near the fornix 132. As described herein, fornix stimulation can effectively treat epileptogenic zones in the hippocampus by overdriving electrical activity at hippocampal epileptic zones electrically associated with the fornix (e.g., connected by fornix neural fibers). The location of the stimulator 134 relative to the fornix 132 may depend, for example, on the location of the epileptogenic focus, seizure frequency and severity or other patient specific parameters.

For example, the stimulator 134 can include one or more electrodes located at the or near the end 136 of the rod 138, which electrodes are operative to provide electrical stimulation according to electrical signals provided by an associated electrical signal generator 142. The signal generator 142 can be programmed and configured to provide electrical signals (e.g., pulses having desired electrical characteristics, such as described hereinabove) to the stimulator 134 for electrically stimulating the fornix 132. Those skilled in the art will understand and appreciate that while a single stimulator rod 138 is illustrated in FIG. 5 that more than one such rod can be utilized for unilateral or bilateral electrical stimulation of the respective fornices. It is further to be appreciated that the electrical stimulation can be implemented as a predetermined schedule (e.g., open loop configuration) or based on one or more sensed conditions (e.g., closed loop configuration).

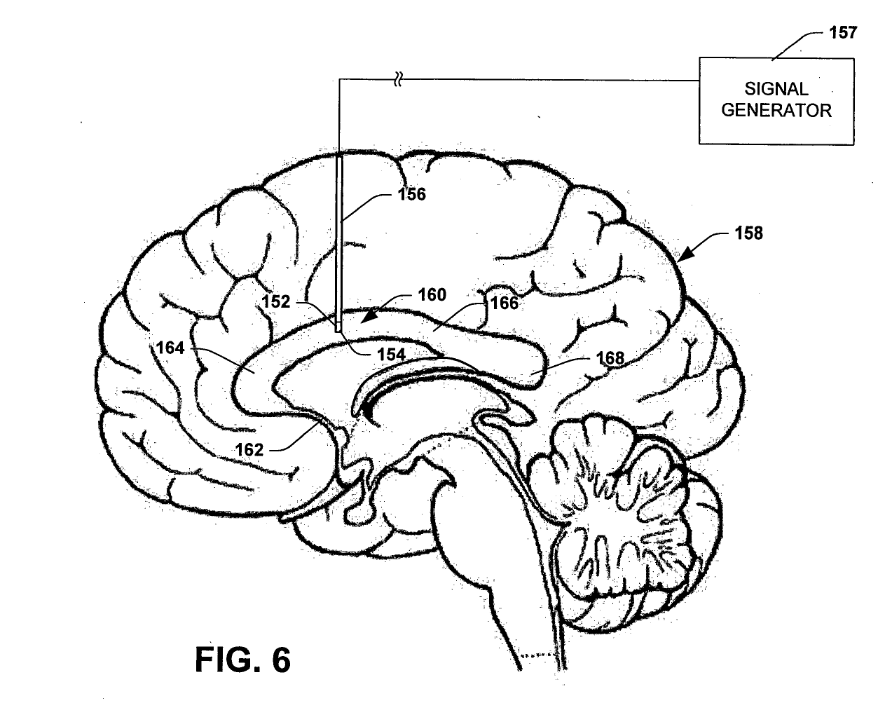

FIG. 6 illustrates an example of corpus callosal stimulation in accordance with an aspect of the present invention. In this example, the type of stimulation system 150 being utilized is similar to that described above with respect to FIG. 5. Briefly stated, the system 150 includes a stimulator 152 that is located at or near an end 154 of an elongated rod 156. The rod is inserted into the brain 158 in a minimally invasive manner to position the stimulator 152 in electrical contact with the corpus callosum 160. The stimulator 152 includes one or more electrodes, which are operative to provide electrical stimulation according to electrical signals provided by an associated electrical signal generator 157. As mentioned above, electrical stimulation of the corpus callosum 160 can effectively treat epileptogenic zones in the neocortical areas by overdriving electrical activity at such epileptic zones.

The corpus callosum 160 is white matter that connects significant regions of the two hemispheres of the brain 158. The corpus callosum 160 includes numerous commissual fibers, specific parts of which interconnect the corpus callosum with corresponding regions of cortex. Various parts of the corpus callosum 160 include the rostrum 162, genu 164, body or trunk 166, and splenium 168. For instance, fibers in the splenium 168 interconnect the occipital and posterior temporal cortices on the two sides of the brain 158. Accordingly, electrical stimulation of selected parts of the corpus callosum 160 can be employed to achieve desired stimulation of correspondingly connected neocortical areas in accordance with an aspect of the present invention. As mentioned above, numerous diagnostic modalities exist for determining the location of one or more epileptogenic foci, such as the cortical areas connected with the corpus callosum 160.

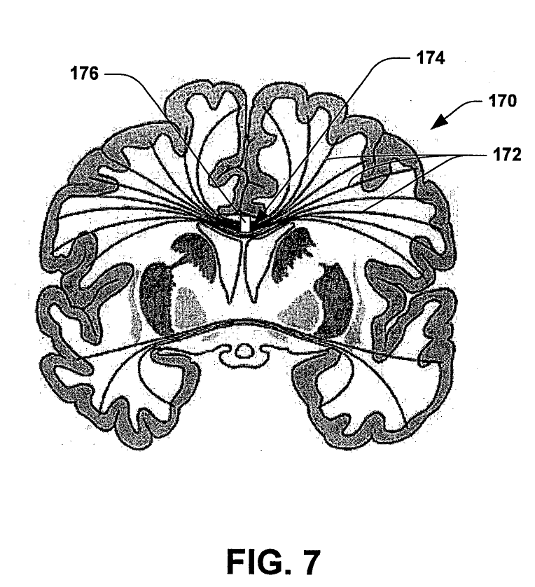

FIG. 7 depicts a coronal section of a brain 170 illustrating white matter fibrous interconnections 172 from the corpus callosum 174 to electrically associated neocortical areas. Thus, by selectively electrically stimulating different parts of the corpus callosum 174 with one or more electrodes 176, corresponding neocortical areas can be effectively electrically stimulated (e.g., overdriven), such as those determined to be epileptogenic zones.

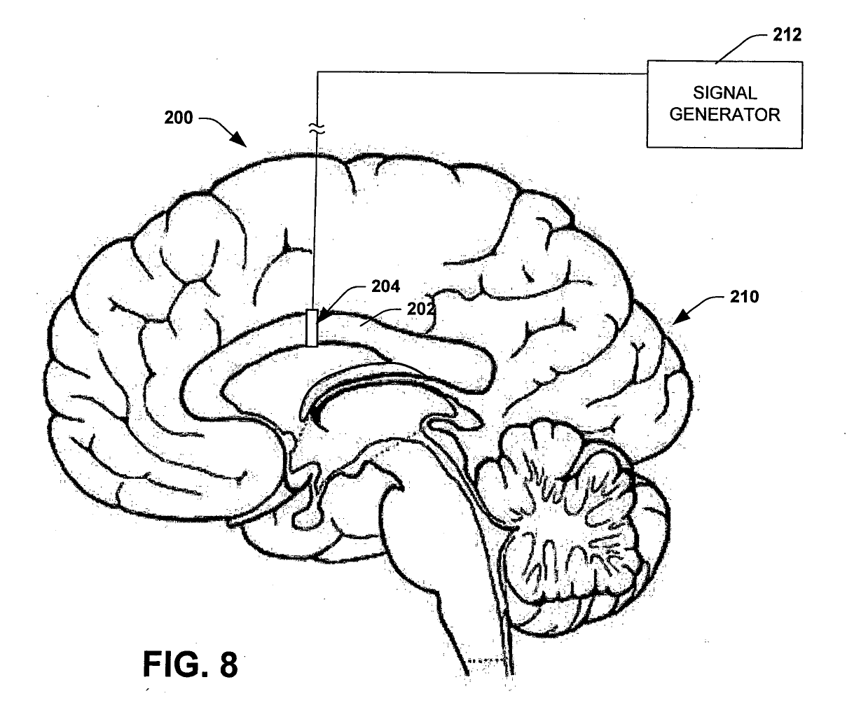

FIG. 8 depicts another type of stimulator system 200 that can be utilized to electrically stimulate the corpus callosum 202 in accordance with an aspect of the present invention. The system 200 includes a stimulator device 204, which in this example is depicted as an implantable electrode device. One or more of such stimulator devices 204 are positioned in electrical contact with selected parts of the corpus callosum 202 associated with cortical areas that have been determined to be epileptogenic zones. Any one or more of the diagnostic modalities mentioned herein above can be employed to ascertain the location of epileptogenic zone or zones for a given patient. The stimulator device 204 can be implanted in the brain 210 using open craniotomy, stereotactic or endoscopic means, for example.

The stimulator device 204 is coupled to a signal generator 212 that is operative to provide electrical signal to the stimulator having desired electrical characteristics, such as described herein. The signal generator 212 can be configured to operate in an open loop manner, thereby providing the electrical signals according to a preprogrammed pulse modulation scheme. Alternatively or additionally, the signal generator can operate in a closed loop manner, such as by generating electrical signal based on one or more sensed conditions. Such sensed conditions can be associated with the epileptogenic zones, for example, signals from sensors indicative of seizure onset. Those skilled in the art will understand and appreciate various algorithms that could be utilized to implement desired stimulation based on, among other things, the patient's condition, severity and frequency of the seizures, location of epileptogenic zones and so forth.

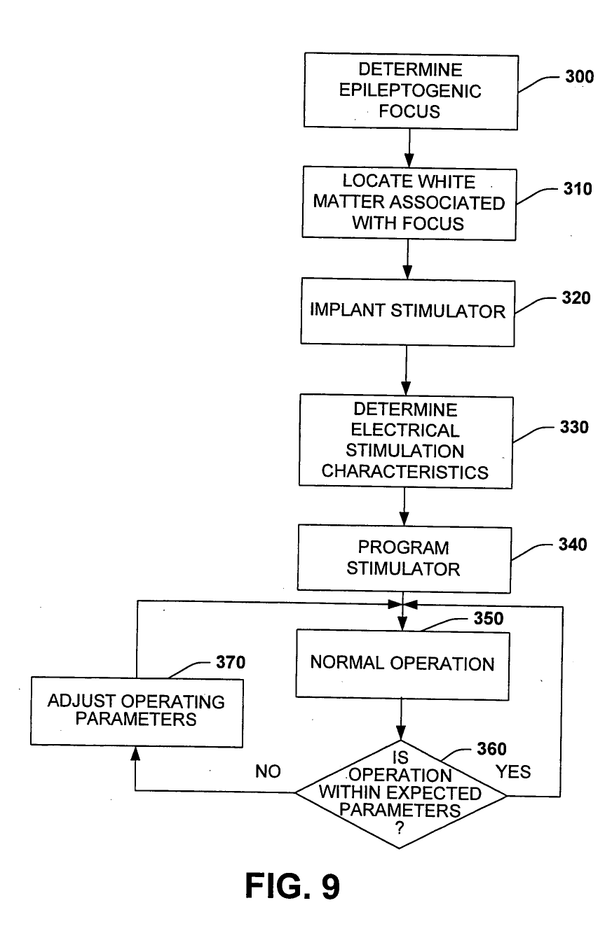

In view of the foregoing structural and functional features described above, a methodology for implementing electrical stimulation of white matter tracts, in accordance with an aspect of the present invention, will be better appreciated with reference to FIG. 9. Those skilled in the art will understand and appreciate that not all illustrated features may be required to implement a methodology in accordance with an aspect of the present invention. While, for purposes of simplicity of explanation, the methodology of FIG. 9 is shown and described as being implemented serially, it is to be understood and appreciated that the present invention is not limited to the illustrated order, as some parts of the methodology could, in accordance with the present invention, occur in different orders or concurrently with other parts from that shown and described. Various parts of the methodology can be implemented as computer executable instructions running in a computer or other microprocessor based device (e.g., a signal generator or other associated control system).

The methodology can be performed for patients that have seizures which are intractable to standard treatments such as various anti-epileptic medications. The methodology begins at 300 in which the location of one or more epileptogenic foci is determined. This determination can be made based on one or a combination of diagnostic modalities, such as mentioned above. Next, at 310 corresponding white matter associated with or otherwise connected with the epileptogenic focus of foci are located. Such locations can define implantation sites for one or more stimulators according to an aspect of the present invention. For example, the fornix can be used if the epileptogenic focus has been determined to be the hippocampus and the corpus callosum can be used for stimulation if the epileptogenic focus has been determined to be neocortical. The implant site can be further selected based on various patient specific parameters, such as mentioned above. Stimulators can be implanted unilaterally or bilaterally depending on the epileptogenic focus or foci.

At 320, a stimulator (or stimulators) is implanted for electrically stimulating the white matter determined at 310. The stimulator can be implanted stereotactically or endoscopically depending generally on the implant site and type of stimulator(s) being used. At 330, electrical stimulation characteristics are determined. The electrical characteristics (e.g., as noted above) generally will vary depending on whether the system is being implemented as an open or closed loop system, a variety or patient specific indications as well as the proximity and electrical pathways interconnecting the white matter and the predetermined epileptogenic zone(s).

At 340, the stimulation system (e.g. signal generator, controls) is programmed to implement desired stimulation of the white matter tract in accordance with an aspect of the present invention. For example, the signal generator can be configured to provide electrical pulses to one or more electrodes of the stimulator at a frequency ranging from about 0.1 Hz to about 5000 Hz. As mentioned above, low frequency, such as less than about 10 Hz (e.g., in a range from about 0.5 Hz to about 4 Hz) can also be employed. The duty cycle of the electrical pulses also can be programmable. The amplitude of electrical current can be set based at least in part on the patient's condition and the white matter structure being stimulated for overdriving the epileptogenic focus. Electrical current pulses can be provided having an amplitude in a range from 0 to about 5 mA, which pulses can be monophasic or polyphasic signals, for example. Normal operation can begin at 350. During normal operation, electrical stimulation of the white matter tract results in indirect electrical stimulation of the determined epileptogenic zone via the electrical pathway provided by the white matter structure fibrously connected with the zone.

At 360, a determination is made as to whether operation of the stimulation system is within expected operating parameters. This determination can be made by physician, such as during seizure monitoring using appropriate diagnostic techniques. Alternatively or additionally, the determination can be made by a processor executing a control program, such as part of a closed loop implementation according to an aspect of the present invention. If the determination is positive, indicating that operation is within expected parameters, the methodology can loop back to 350 and continue normal operation. If the determination is negative, the methodology proceeds to 370 in which one or more operating parameters can be adjusted. Such adjustments can be made manually by physician (e.g., reprogramming the stimulation system) to optimize operation for mitigating or helping control seizures for the patient. The adjustments can be based on empirical studies and other data (e.g., patient-specific data or aggregate data collected from a group of patients). Those skilled in the art will understand and appreciate that such adjustments also can be implemented in real time, such as part of the closed loop control process based on feedback from one or more sensors (e.g., intra-cranial or external). The adjustments can also include stopping stimulations for an extended period of time or indefinitely, if deemed appropriate.

From 370, the methodology returns to 350 in which normal operation can continue based on the adjustments at 370.

Appendix A, which forms an integral part of the subject application, includes additional information related to brain stimulation in accordance with one or more aspects of the present invention.

What has been described above includes examples and implementations of the present invention. Because it is not possible to describe every conceivable combination of components, circuitry or methodologies for purposes of describing the present invention, one of ordinary skill in the art will recognize that many further combinations and permutations of the present invention are possible.

For example, brain stimulation according to an aspect of the present invention can be combined with other treatment modalities (e.g., chemical stimulation, drug therapy). In particular, those skilled in the art will understand and appreciate that the connections to the stimulator could include means for administering chemicals (e.g., catheterization coupled to a source of chemicals and associated pumping mechanism) and that the stimulator itself can be implemented as a chemical delivery device to provide corresponding chemical stimulation. It further is to be appreciated that the administration of chemicals to stimulate white matter can be implemented individually, such as an alternative to electrical stimulation, or it can be combined with electrical stimulation of white matter structures in accordance with an aspect of the present invention. When electrical and chemical treatments are combined, the same or different delivery mechanisms can be employed to administer the respective chemical and electrical stimulation according to an aspect of the present invention.

Additionally, while the above description has primarily dealt with treating epileptic seizures, those skilled in the art will understand that it is equally applicable to other types of degenerative diseases and nervous system disorders, such as Parkinson's disease.

In view of the foregoing, the present invention is intended to embrace all such alterations, modifications and variations that fall within the spirit and scope of the appended claims.

1. A brain stimulation system, comprising:
a stimulator operative to electrically stimulate a white matter brain structure associated with a non-white matter brain structure, whereby stimulation of the white matter brain structure can overdrive at least some electrical activity of the non-white matter brain structure.

2. The system of claim 1, the non-white matter brain structure further comprising a predetermined epileptogenic zone fibrously connected with the white matter brain structure.

3. The system of claim 1, the predetermined epileptogenic zone comprising at least one of hippocampus, cortical structure and temporal lobe.

4. The system of claim 3, the white matter brain structure comprising at least one of fornix, corpus callosum and temporal stem white matter, according to the predetermined epileptogenic zone.

5. The system of claim 1, further comprising a signal generator operative to cause the stimulator to provide an electrical signal having an electrical characteristic for stimulating the white matter brain structure.

6. The system of claim 5, the electrical characteristic further comprising a frequency that is less than about 10 Hz.

7. The system of claim 6, the frequency being less than about 3 Hz.

8. The system of claim 1, the stimulator comprising a generally cylindrical body portion having at least one electrode located at an interior portion of the body portion for electrically stimulating the white matter brain structure based on a signal from an associated control system.

9. The system of claim 8, the generally cylindrical body portion having an annular or generally C-shaped cross section, an inner sidewall portion of which is dimensioned and configured for attachment to the fornix.

10. A method of brain stimulation, comprising:
placing at least one electrode at a location for electrically stimulating a white matter brain structure; and
using the at least one electrode to electrically stimulate the white matter brain structure to overdrive at least some electrical activity of a non-white matter brain structure associated with the white matter brain structure.

11. The method of claim 10, further comprising determining a location of the non-white matter brain structure.

12. The method of claim 1 1, the non-white matter brain structure further comprising an epileptogenic zone.

13. The method of claim 12, the epileptogenic zone comprising at least one of a hippocampus, cortical structure and temporal lobe.

14. The method of claim 12, further comprising implanting the electrode for electrically stimulating at least one of the fornix, corpus callosum and temporal stem based on the determined epileptogenic zone.

15. The method of claim 10, the white matter brain structure further comprising at least one of the fornix, corpus callosum and temporal stem white matter.

16. The method of claim 1, further comprising causing the electrode to electrically stimulate the white matter brain structure with an electrical signal having an electrical characteristic.

17. The method of claim 16, the electrical characteristic further comprising a frequency that is less than about 10 Hz.

18. The method of claim 17, the frequency being less than or equal to about 3 Hz.

19. The method of claim 10, the placement of the at least one electrode further comprising substantially securing the at least one electrode in electrical contact with the white matter brain structure to facilitate electrical stimulation thereof.

20. The method of claim 10, the placement of the at least one electrode further comprising endoscopy to facilitate placing the at least one electrode in electrical contact with the white matter brain structure.
